(19) United States
(12) Patent Application Publication
(10) Pub. No.: US 2010/0069617 A1
(43) Pub. Date: Mar. 18, 2010

(54) ENHANCED PROTEIN AGGREGATE REMOVAL BY MIXED MODE CHROMATOGRAPHY ON HYDROPHOBIC INTERACTION MEDIA IN THE PRESENCE OF PROTEIN-EXCLUDED ZWITTERIONS

(75) Inventor: PETER S. GAGNON, San Clemente, CA (US)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA, SE (unknown)

(21) Appl. No.: 12/553,466

(22) Filed: Sep. 3, 2009

Related U.S. Application Data
(60) Provisional application No. 61/191,780, filed on Sep. 12, 2008.

(51) Int. Cl. C07K 1/14

(52) U.S. Cl. 530/413; 530/415

(57) ABSTRACT

This invention relates to methods for enhancing purification of proteins such as monoclonal antibodies by chromatography on carboxyl group-containing HIC supports in the presence of zwitterions that are excluded from protein surfaces. In certain embodiments, the invention may permit more effective separation of non-aggregated protein from aggregated protein.

### CROSS-REFERENCE TO RELATED APPLICATIONS

This application claims priority to U.S. provisional patent application No. 61/191,780 filed Sep. 12, 2008; the entire disclosure is incorporated herein by reference.

### FIELD OF THE INVENTION

This invention relates to methods for enhancing separation of proteins such as monoclonal antibodies by chromatography on HIC supports in the presence of zwitterions that are excluded from protein surfaces. In certain embodiments, the invention may permit more effective separation of non-aggregated protein from aggregated protein.

### BACKGROUND OF THE INVENTION

HIC is a well-established method for separation of proteins, including antibodies. Hydrophobic surface residues on proteins interact with hydrophobic ligands on a chromatography support. Proteins with more hydrophobic groups are retained more strongly than proteins with fewer hydrophobic groups. Under appropriate conditions, bound proteins elute in order of increasing hydrophobicity. The implication is that HIC should be effective for aggregate removal since aggregates represent multiples of the native protein, potentially with many more hydrophobic residues to mediate binding. Aggregation is also associated frequently with damage to a given protein, which may lead to surface exposure of hydrophobic residues that are normally internalized within the structure, thereby further enhancing aggregate binding. Through either or both mechanisms, HIC may thus be used to reduce aggregate content of protein preparations, however it frequently fails to do so with adequate efficiency to support commercial applications.

Selectivity in HIC is most commonly controlled with solutions of salts that are known to promote hydrophobic interactions. Examples include ammonium sulfate, sodium sulfate, potassium phosphate, sodium citrate, and sodium chloride. Proteins are bound at a high concentration of one of these salts, then eluted in a descending gradient of the same salt. A general liability of these applications is that antibodies often elute at high salt concentrations that require dilution or buffer exchange before they can be processed by other chromatography methods such as ion exchange. This burdens process logistics and economics. This limitation can be overcome by binding the antibody in glycine and eluting it in a descending glycine gradient. In the pH range where glycine is zwitterionic, it does not contribute to conductivity, which means that it does not interfere with downstream ion exchange chromatography steps (Gagnon, 2000, The use of hydrophobic interaction chromatography with a non-salt buffer system for improving process economics in purification of monoclonal antibodies, oral presentation, Waterside Conference, Miami, April 30-May 3).

Some HIC supports contain carboxyl groups capable of mediating cation exchange interactions. This creates the possibility of mixed mode interactions between proteins and such a chromatography support. More specifically, it creates the possibility of using low pH to promote protein retention and high pH to weaken protein retention. High salt concentrations suppress charge interactions however. In order for pH to substantially affect selectivity, the conductivity must be low.

The addition of 1.75 M glycine to an antibody sample in HIC separations on Tosoh Butyl 600 M and Tosoh Phenyl 750 M media has been described (J. F. Kramarczyk, et al: Biotech Bioeng 100, 707-720 (2008)). There is however a risk for precipitation when high concentrations of additives are applied to protein solutions.

### SUMMARY OF THE INVENTION

The present invention relates to a method of separating at least one non-aggregated protein from a liquid preparation by contacting said preparation with a carboxyl-containing HIC support at a conductivity of less than 25 mS/cm in the presence of one or more species of protein-excluded zwitterions at a combined concentration greater than 0.5 M. Applicant surprisingly found that low-conductivity mixed mode interactions on carboxyl-containing HIC supports in the presence of protein-excluded zwitterions, permit more effective aggregate removal than conventional HIC elution methods.

Alternatively, the non-aggregated proteins are separated from a liquid preparation by contacting said preparation with a carboxyl-containing HIC support and subsequently contacting said support with a liquid having conductivity of less than 25 mS/cm and a concentration of protein-excluded zwitterions greater than 0.5 M.

In some embodiments, practicing the invention may permit removal of aggregates to lower levels than can be achieved in the absence of the invention.

In some embodiments, practicing the invention may permit effective aggregate removal from a larger amount protein, as measured in mg of protein per mL of carboxyl-containing HIC media, than can be achieved in the absence of the invention.

In some embodiments, practicing the invention may permit aggregate removal to a lower level and from a larger amount of protein than can be achieved in the absence of the invention.

In some embodiments, practicing the invention may permit aggregates to be removed effectively from protein preparations that cannot be accommodated in the absence of the invention.

In some embodiments, the protein preparation may be applied to the carboxyl-containing HIC-support under conditions that permit the binding of nonaggregated protein and aggregated protein, with separation of non-aggregated protein being achieved subsequently by application of an elution gradient. This mode of chromatography is often referred to as bind-elute mode.

In some embodiments, the protein preparation may be applied to the carboxyl-containing HIC-support under conditions that prevent the binding of intact nonaggregated protein, while selectively binding or retaining aggregates. This mode of application is often referred to as flow-though mode. Bound aggregates may be removed subsequently from the column by means of a cleaning step.

The invention may be practiced in combination with one or more other separation methods.

Additional objects and advantages of the invention will be set forth in part in the description which follows, and in part will be obvious from the description, or may be learned by practice of the invention. The objects and advantages of the invention will be realized and attained by means of the elements and combinations specified in the claims.

It is to be understood that both the foregoing general description and the following detailed description are exemplary and explanatory only and are not restrictive of the invention, as claimed.

### DETAILED DESCRIPTION OF THE INVENTION

### Definitions

Terms are defined so that the invention may be understood more readily. Additional definitions are set forth throughout the detailed description.

“Carboxyl-containing HIC support” refers to a chromatography support intended for performing the technique of HIC, which contains carboxyl groups capable of mediating cation exchange interactions with proteins. Examples include but are not limited to carboxyl-containing HIC supports that employ aliphatic hydrophobic ligands such as butyl or hexyl, or aromatic hydrophobic ligands such as phenyl. The carboxyl-containing HIC supports can either a) have carboxyl groups intentionally introduced, e.g. by derivatization with carboxymethyl groups, or b) the carboxyl groups can be inherently present due to the chemistries used in the manufacture of the support. Such inherent carboxyls are present in many methacrylate and acrylamide derived supports due to partial hydrolysis of the ester/amide groups in the base matrix. Polysaccharide media may also show some carboxyl groups due to oxidation of aldehydes.

“Salt” refers to an aqueous-soluble ionic compound formed by the combination of negatively charged anions and positively charged cations. The anion or cation may be of organic or inorganic origin. Examples include but are not limited to sodium chloride.

“Protein-excluded zwitterions” refers to molecules with molecular weights less than 1000 daltons that bear a balance of positive and negative charges at a given pH such that they do not contribute significantly to the conductivity of the solution in which they are dissolved. Such substances are generally characterized by high molar dielectric increments and positive surface tension increments. The term “protein-excluded” arises from the fact that these molecules are repelled from protein surfaces. Examples of protein-excluded zwitterions may include naturally occurring, chemically modified, or exclusively synthetic compositions, including but not limited to glycine, betaine, taurine, tauro-betaine, morpholinoethanesulfonic acid (MES), hydroxyethylpiperazinesulfonic acid (HEPES), and N,N-Bis(2-hydroxyethyl)glycine (BICINE).

“Buffering compound” refers to a chemical compound employed for the purpose of stabilizing the pH of an aqueous solution within a specified range. Phosphate is one example of a buffering compound. Other common examples include but are not limited to compounds such as acetate, citrate, borate, MES, Tris, HEPES, and BICINE.

“Buffer” refers to an aqueous formulation comprising a buffering compound and other components required to establish a specified set of conditions to mediate control of a chromatography support. The term “equilibration buffer” refers to a buffer formulated to create the initial operating conditions for a chromatographic operation. “Wash buffer” refers to a buffer formulated to displace unbound contaminants from a chromatography support. “Elution buffer” refers to a buffer formulated to displace proteins from the chromatography support.

“Protein” refers to any type of protein, glycoprotein, phosphoprotein or protein conjugate. Antibodies constitute a commercially important class of proteins. Other proteins of commercial interest are peptides, insulin, erythropoietin, interferons, enzymes, plasma proteins etc. The molecules of the protein may comprise several subunit chains joined together by e.g. disulfide bonds. In the case of proteins naturally occurring as multimers, each such multimer is here considered as a protein molecule.

“Antibody” refers to any immunoglobulin, composite, or fragmentary form thereof. The term may include, but is not limited to polyclonal or monoclonal antibodies of the classes IgA, IgD, IgE, IgG, and IgM, derived from human or other mammalian cell lines, including natural or genetically modified forms such as humanized, human, chimeric, synthetic, recombinant, hybrid, mutated, grafted, and in vitro generated antibodies.

“Antibodies” may also include composite forms including but not limited to fusion proteins containing an immunoglobulin moiety. “Antibodies” may also include fragmentary forms such as single chain antibodies, Fab, F(ab′)2, Fv, scFv, Fd, mAb, dAb or other compositions that retain antigen-binding function.

“Protein aggregate” refers to an association of at least two protein molecules. The association may be either covalent or non-covalent without respect to the mechanism by which the protein molecules are associated. The association may be direct between the protein molecules or indirect through other molecules that link the protein molecules together. Examples of the latter include but are not limited to disulfide linkages with other proteins, hydrophobic associations with lipids, charge associations with DNA, affinity associations with leached protein A, or mixed mode associations with multiple components.

“Protein preparation” refers to any composition containing a non-aggregated protein (e.g. an IgG antibody). Said preparation may contain protein fragments and/or aggregates. Other proteins and other contaminants, potentially including but not limited to nucleic acids, endotoxins, and virus particles may also be present.

As it relates to the invention herein, the term “bind-elute mode” refers to an operational approach to chromatography in which the buffer conditions are established so that the non-aggregated protein and some contaminants bind to the column upon application, with fractionation being achieved subsequently by modification of the buffer conditions. Fractionation is most commonly achieved by applying an elution gradient, in which the concentration of one or more buffer components, or conditions such as pH, are increased or decreased. The increase or decrease may be essentially continuous, as in the case of so-called linear gradients, or incremental, as in the case of so-called step gradients.

As it relates to the invention herein, the term “flow-through mode” refers to an operational approach to chromatography in which the buffer conditions are established so that the non-aggregated protein flows through the column upon application while contaminants are selectively bound or retained, thus achieving their removal.

“Preparative applications” refers to situations in which the invention is practiced for the purpose of separating non-aggregated protein for research, diagnostic, or therapeutic applications. Such applications may be practiced at any scale, ranging from milligrams to kilograms of protein per batch.

### Carboxyl-Containing HIC Supports

The invention may be practiced with any of a large number of carboxyl-containing HIC supports, including but not limited to a) supports prepared from methacrylate base matrices such as Butyl Toyopearl, Hexyl Toyopearl, Phenyl Toyopearl (Tosoh Bioscience LLC), FRACTOGEL® EMD Propyl, FRACTOGEL® EMD Phenyl (Merck Chemicals Darmstadt), MACRO-PREP® Methyl or MACRO-PREP® t-Butyl (Bio-Rad Laboratories, Inc.), of any particle size or pore size distribution or b) supports where carboxyl groups have been deliberately introduced by e.g. carboxymethylation (reaction with monochloroacetic acid or monobromoacetic acid), oxidation of aldehydes or any other methods known in the art. The number of carboxyl groups can be from 0.1 to 200 micromol/ml support, such as 1 to 100, 1 to 50 or 2 to 20 micromol/ml. The number of carboxyl groups can be determined primarily by H⁺ capacity titration or alternatively by spectroscopic methods, both of which are well known in the art. The hydrophobic groups in the carboxyl-containing HIC supports may be e.g. ethyl, butyl, hexyl, octyl or phenyl groups.

The invention may be practiced in a packed bed column, a fluidized/expanded bed column containing the HIC support and/or a batch operation where the HIC support is mixed with the solution for a certain time.

One embodiment employs a HIC support packed in a column.

One embodiment employs a HIC support, packed in a column of about 5-10 mm internal diameter and a height of about 5-50 mm, for evaluating the effects of various buffer conditions on the binding and elution characteristics of a particular protein preparation or protein fragment preparation. The column may comprise HIC support packed in one or more wells of a multiwell filter plate.

Another embodiment employs a HIC support, packed in columns of any dimensions required to support preparative applications. Column diameter may range from 1 cm to more than 1 meter, and column height may range from 5 cm to more than 30 cm depending on the requirements of a particular application.

Appropriate column dimensions can be determined by the skilled artisan.

In one embodiment, the carboxyl-containing HIC support is in the form of a membrane, optionally accommodated in a membrane adsorber device.

### Protein Preparations

Protein preparations to which the invention can be applied may include unpurified or partially purified proteins from natural, synthetic, or recombinant sources. Unpurified protein preparations may come from various sources including, but not limited to, plasma, serum, ascites fluid, milk, plant extracts, bacterial lysates, yeast lysates, or conditioned cell culture media. Partially purified preparations may come from unpurified preparations that have been processed by at least one chromatography, precipitation, other fractionation step, or any combination of the foregoing. The chromatography step or steps may employ any method, including but not limited to size exclusion, affinity, anion exchange, cation exchange, protein A affinity, hydrophobic interaction, immobilized metal affinity chromatography, or hydroxyapatite chromatography. The precipitation step or steps may include salt or PEG precipitation, or precipitation with organic acids, organic bases, or other agents. Other fractionation steps may include but are not limited to crystallization, liquid:liquid partitioning, or membrane filtration.

In one embodiment, the protein comprises antibodies, such as e.g. IgG antibodies.

### Description of the Method

In preparation for contacting the protein preparation with the HIC column, it is usually necessary to equilibrate the chemical environment inside the column. This is accomplished by flowing an equilibration buffer through the column to establish the appropriate pH, conductivity, concentration of salts; and/or the identity and concentration of protein-excluded zwitterions.

The equilibration buffer for applications conducted in bind-elute mode may include any of a wide range of options depending on the binding requirements of a particular protein. The equilibration buffer will normally include a buffering compound to confer adequate pH control. Buffering compounds may include but are not limited to MES, HEPES, BICINE, imidazole, Tris, phosphate, citrate, or acetate, or some mixture of the foregoing. The concentration of the buffering compound in an equilibration buffer commonly ranges from 10 to 50 mM. The equilibration buffer will usually contain a salt or (optionally) protein-excluded zwitterions at a concentration greater than 1 M to promote protein binding. The pH of the equilibration buffer may range from about pH 4.5 to pH 9.0. In one embodiment, the equilibration buffer has a conductivity of less than 25 mS/cm and a concentration greater than 0.5 M of protein-excluded zwitterions. In further embodiments, the conductivity is less than 20 mS/cm or 15 mS/cm and/or the concentration of protein-excluded zwitterions is greater than 1 M, 1.5 M or 1.8 M. In one embodiment, the equilibration buffer contains less than 0.5 M protein-excluded zwitterions to avoid precipitation phenomena.

The protein preparation may also be equilibrated to conditions compatible with the column equilibration buffer in order to facilitate effective binding. In one embodiment, the protein preparation has a conductivity of less than 25 mS/cm and a concentration greater than 0.5 M of protein-excluded zwitterions. In further embodiments, the conductivity is less than 20 mS/cm or 15 mS/cm and/or the concentration of protein-excluded zwitterions is greater than 1 M, 1.5 M or 1.8 M. In one embodiment, the protein preparation contains less than 0.5 M protein-excluded zwitterions to avoid precipitation phenomena.

After the column and protein preparation have been equilibrated, the protein preparation may be contacted with the column. It may be applied at a linear flow velocity in the range of, but not limited to, about 50-600 cm/hr. The appropriate flow velocity can be determined by the skilled artisan.

After the sample has been applied, the column is washed to remove unbound contaminants. In one embodiment, the wash buffer has a conductivity of less than 25 mS/cm and a concentration greater than 0.5 M of protein-excluded zwitterions. In further embodiments, the conductivity is less than 20 mS/cm or 15 mS/cm and/or the concentration of protein-excluded zwitterions is greater than 1 M, 1.5 M or 1.8 M. In one embodiment protein-excluded zwitterion at a concentration exceeding 0.5 M is added first during the washing step, to reduce the risk of precipitation.

The wash buffer may serve the additional purpose of re-equilibrating the column to conditions suitable for practicing the invention.

Following the wash step, non-aggregated protein is selectively eluted from the column. In one embodiment, the elution buffer at some point during elution has a conductivity of less than 25 mS/cm and a concentration greater than 0.5 M of protein-excluded zwitterions. In further embodiments, the conductivity is at some point less than 20 mS/cm or 15 mS/cm and/or the concentration of protein-excluded zwitterions is greater than 1 M, 1.5 M or 1.8 M.

In one embodiment, the concentration of protein-excluded zwitterions is increased during elution.

In one embodiment, the concentration of protein-excluded zwitterions is constant during elution.

In one embodiment, the concentration of protein-excluded zwitterions is decreased during elution.

The useful pH range of a particular compound for practicing the invention is determined by its pH titration characteristics. Glycine is in its zwitterionic form at pH values ranging from about pH 4 to about pH 8. Betaine is in its zwitterionic form at pH values ranging from about pH 4 to about pH 10. Taurine is in its zwitterionic form at pH values ranging from about pH 3 to about pH 8. In one embodiment the pH value is chosen so that the compound is in its zwitterionic form.

In one embodiment the protein-excluded zwitterion is selected from the group consisting of glycine, betaine, taurine, tauro-betaine, MES, HEPES and BICINE. In another embodiment the protein-excluded zwitterion is selected from the group consisting of betaine, taurine, tauro-betaine, MES, HEPES and BICINE.

After elution, the column may optionally be cleaned and re-used.

In one embodiment of the flow-through mode, the non-aggregated protein flows through the column and is collected, while aggregated protein binds to the column. The protein preparation is followed with a wash buffer, usually of the same composition as the equilibration buffer. This displaces remaining non-aggregated protein from the column so that it can be collected. Bound contaminants may optionally be removed from the column with a cleaning buffer. The column may optionally be re-used, or stored in an appropriate agent for later re-use.

It will be apparent to the person of ordinary skill that the invention may have a beneficial effect on removal of other contaminants, such as contaminant proteins (e.g. host cell proteins), nucleic acids, endotoxins, virus particles, and complexes of antibody with leached protein A.

### Additional Optional Steps

The present invention may be combined with other separation methods to achieve higher levels of purification, if necessary. The invention may be practiced at any point in a sequence of 2 or more separation methods. Examples of methods include, but are not limited to, other methods commonly used for purification of proteins (e.g. antibodies), such as size exclusion chromatography, protein A and other forms of affinity chromatography, anion exchange chromatography, cation exchange chromatography, hydrophobic interaction chromatography, immobilized metal affinity chromatography, hydroxyapatite chromatography, precipitation, crystallization, liquid:liquid partitioning, and various filtration methods. In a specific embodiment, a protein preparation previously purified on a protein A column is contacted with a carboxyl-containing HIC support at a conductivity of less than 25 mS/cm in the presence of protein-excluded zwitterions at a concentration greater than 0.5 M. In a further specific embodiment, a protein preparation is first contacted with a carboxyl-containing HIC support in the presence of protein-excluded zwitterions and afterwards contacted with an ion exchange medium. It is within the purview of one of ordinary skill in the art to develop appropriate conditions for the various methods and integrate them with the invention herein to achieve the necessary purification of a particular protein.

It is well known in the art of protein separation that considerable variation in chromatographic behavior is encountered from one protein preparation to another. This includes variation in the composition and proportion of contaminant proteins, intact target protein, target protein fragments, and protein aggregates that reside within various preparations, as well as variation in the individual retention characteristics of different constituents. This makes it necessary to customize the buffer conditions to apply the invention to its best advantage in each situation. This may involve adjustment of pH, the concentration of salts, the concentration of buffering components, and identity and content of the protein-excluded zwitterion. Appropriate levels for the various parameters and components can be determined systematically by a variety of approaches. The following examples are offered for illustrative purposes only.

### Examples

Below the present invention will be disclosed by way of examples, which are intended solely for illustrative purposes and should not be construed as limiting the present invention as defined in the appended claims. All references mentioned below or elsewhere in the present application are hereby included by reference.

### Example 1

A 1 mL column with dimensions of 5×50 mm is packed with Phenyl Toyopearl 650M and equilibrated to 20 mM MES, 2.0 M sodium chloride, pH 6.0, at a flow rate of 1 mL/min (300 cm/hr). 20 mg of protein A purified antibody at column equilibration conditions is injected. The column is washed with 20 mM MES, 2.0 M glycine, 20 mM sodium chloride, pH 6.0, to displace contaminants. The column is eluted in a 20 column volume (CV) linear gradient to 20 mM Tris, 20 mM Hepes, 20 mM MES, 20 mM sodium chloride, 1.0 M glycine, pH 8.0.

### Example 2

A 1 mL column with dimensions of 5×50 mm is packed with Butyl Toyopearl 650M and equilibrated to 20 M sodium citrate, 2.0 M sodium chloride pH 5.0, at a flow rate of 1 mL/min (300 cm/hr). 20 mg of protein A purified antibody at column equilibration conditions is injected. The column is washed with 20 mM citrate, 2.0 M glycine, pH 5.0 to displace contaminants. A second wash is applied to re-equilibrate the column to 20 mM citrate, 1.0 M glycine, pH 5.0, leaving the antibody retained largely by cation exchange. The column is eluted in a 20 CV linear gradient to 20 mM citrate, 20 mM phosphate, 20 mM citrate, 1.0 M glycine, pH 7.5.

It will be understood by the person of ordinary skill in the art how to convert bind elute conditions to flow-through conditions, and optimize and scale up the results from experiments such as those described in the above examples. It will also be understood by such persons that other approaches to method development, such as but not limited to high-throughput robotic systems, can be employed to determine the conditions that most effectively embody the invention for a particular protein.

All cited references are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, chromatography conditions, and so forth used in the specification and claims are to be understood as being modified in all instances by the term “about.” Accordingly, unless indicated to the contrary, the numerical parameters set forth in the specification and attached claims are approximations that may vary depending upon the desired performance sought to be obtained by the present invention.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

What is claimed is:

1. A method for separating at least one non-aggregated protein from a liquid preparation comprising contacting said preparation with a carboxyl-containing HIC support at a conductivity of less than 25 mS/cm in the presence of protein-excluded zwitterions at a concentration greater than 0.5 M.

2. A method for separating at least one non-aggregated protein from a liquid preparation comprising contacting said preparation with a carboxyl-containing HIC support and subsequently contacting said support with a liquid having conductivity of less than 25 mS/cm and a concentration of protein-excluded zwitterions greater than 0.5 M.

3. The method of claim 1, wherein said protein is an antibody.

4. The method of claim 3, wherein the antibody is of the class IgA, IgD, IgE, IgG, or IgM.

5. The method of claim 3, wherein the antibody is a fragment that retains the ability to bind antigen.

6. The method of claim 3, wherein the antibody is a fusion protein containing an antibody moiety.

7. The method of claim 1, wherein the protein-excluded zwitterions is glycine, betaine, or taurine.

8. The method of claim 1, wherein the protein-excluded zwitterions is betaine, taurine, tauro-betaine, morpholinoethanesulfonic acid (MES), hydroxyethylpiperazinesulfonic acid (HEPES), or N,N-Bis(2-hydroxyethyl)glycine (BICINE).

9. The method of claim 1, wherein the hydrophobic ligand on the carboxyl-containing HIC support is butyl or phenyl.

10. The method of claim 1, wherein the method is combined with other separation methods to create a multistep purification process.

11. The method of claim 2, wherein said protein is an antibody.

12. The method of claim 11, wherein the antibody is of the class IgA, IgD, IgE, IgG, or IgM.

13. The method of claim 11, wherein the antibody is a fragment that retains the ability to bind antigen.

14. The method of claim 11, wherein the antibody is a fusion protein containing an antibody moiety.

15. The method of claim 2, wherein the protein-excluded zwitterions is glycine, betaine, or taurine.

16. The method of claim 2, wherein the protein-excluded zwitterions is betaine, taurine, tauro-betaine, morpholinoethanesulfonic acid (MES), hydroxyethylpiperazinesulfonic acid (HEPES), or N,N-Bis(2-hydroxyethyl)glycine (BICINE).

17. The method of claim 2, wherein the hydrophobic ligand on the carboxyl-containing HIC support is butyl or phenyl.

18. The method of claim 2, wherein the method is combined with other separation methods to create a multistep purification process.
